(19) United States
(12) Patent Application Publication
(10) Pub. No.: US 2016/0296161 A1
(43) Pub. Date: Oct. 13, 2016

(54) DEVICES AND METHODS FOR ASSESSING AND PROMOTING ORAL MOTOR PERFORMANCE

(71) Applicant: JONATHAN WALLER, Sherman Oaks, CA (US)

(72) Inventor: JONATHAN WALLER, Sherman Oaks, CA (US)

(21) Appl. No.: 14/680,950

(22) Filed: Apr. 7, 2015

(51) Int. Cl. A61B 5/00; A61B 5/11

(52) U.S. Cl. CPC A61B 5/4542; A61B 5/1126; A61B 5/0002; A61B 5/742

(57) ABSTRACT

Systems and methods for obtaining an indication of an oral motor performance that utilize a pressure transducer configured to be positioned within a user's mouth and adapted to generate a transducer signal in response to pressure, a processor adapted to receive and process the transducer signal, and a mobile device adapted to receive the processed transducer signal and display corresponding pressure unit of the processed transducer signal.

### CROSS-REFERENCE TO RELATED APPLICATIONS

This application claims the benefit of U.S. Provisional Application No. 61/977,546 filed Apr. 9, 2014, the disclosure of which is incorporated in its entirety by reference herein.

### TECHNICAL FIELD

The present disclosure relates to devices and methods for assessing and promoting oral motor performance.

### BACKGROUND

Difficulty in swallowing is commonly referred to as oropharyngeal dysphagia. Oropharyngeal dysphagia is often associated with symptoms that include difficulty controlling food orally, difficulty managing saliva, difficulty initiating a swallow, coughing, choking, unintentional weight loss, recurrent pneumonia and feelings of food getting stuck. These symptoms of oropharyngeal dysphagia are common in people with stroke, traumatic brain injury, head and neck cancer and progressive neurological diseases. Assessing, monitoring, and promoting the strength and endurance of the tongue are keys to preventing or treating dysphagia. The tongue plays a major role in the swallowing process, as it transports food toward the pharynx and the esophagus.

A device called the Iowa Oral Performance Instrument (or the IOPI® System) is currently being used to measure the strength and endurance of the tongue and the lip. The IOPI System includes a tongue bulb that is connected to an IOPI module by a connecting tube. The tongue bulb is to be placed in the patient's mouth. The IOPI module includes a display bfor pressure readings and reports pressure in kPA units. It also has a timer for measuring tongue endurance and several lights to indicate the strength of the pressure being applied by the tongue on the tongue bulb. One problem with the IOPI System is it lacks the ability to store and process the data that it reports. The user of the IOPI system has to write down the pressure readings and the time measurement for each test, or enter them into a computer to save them. In a medical office setting involving multiple patients, one would have to retrieve the physical or electronic file of each patient, which is separate from the IOPI system, supplement each file with the results, and individually file them. The patient file cannot also be readily shared or distributed.

The above problems and other problems are addressed by this disclosure as summarized below.

### SUMMARY

The present disclosure relates to a system for obtaining an indication of a patient's oral motor performance. The system includes a pressure transducer adapted to generate a transducer signal after receiving pressure from a part of a patient's mouth and a processor adapted to receive and process the transducer signal. The system also includes a mobile device in communication with the processor adapted to receive the processed transducer signal and display corresponding pressure unit of the processed transducer signal. In one embodiment, the system includes a Bluetooth module connected with the processor and adapted to send the processed transducer signal to the mobile device via airwaves. In another embodiment, the mobile device receives the processed transducer signal through an audio jack. In yet another embodiment, the system includes an analog input/output server adapted to receive the transducer signal from the pressure transducer and transmit the transducer signal to a network. The mobile device receives the transducer signal through the network. The mobile device may be a smartphone or a computer, including a desktop or a mobile computer.

The system may also include a user interface provided by a software program or control logic executed by a microprocessor and presented to a user. The user interface may be configured to control the data collection process in cooperation with the transducer. In various embodiments, the user interface may also be used to provide feedback to a user during a diagnostic test using the transducer. Feedback may include visual, audio, or tactile feedback (such as vibration) to initiate a test, at the conclusion of a data gathering operation, or during data gathering in response to the transducer signal exceeding a threshold, for example. The user interface may also be used to initiate processing of the transducer data, or transmit the transducer data over a network for storage and/or subsequent processing. Test results may be visually displayed to the user during the test and/or subsequent to processing of the transducer signal data using various types of audio signals, visual displays, or simulated speech, for example.

The present disclosure also relates to an oral motor performance assessment and therapy device. The device includes a pressure transducer adapted to obtain a pressure signal from a patient's mouth. The pressure transducer is connected to a processor, which is adapted to obtain the pressure signal from the pressure transducer and convert the pressure signal to a digital pressure value. An audio plug is connected to the processor and is adapted to transmit the digital pressure value to a smartphone. The smartphone is connected to the audio plug and is configured to display and store the digital pressure value. The digital pressure value may be transmitted through a microphone input of the smartphone. The processor preferably operates with a sampling rate of at least 20 kHz. In other embodiments, the device includes a step up transformer connected with the audio plug adapted to step up voltage of the audio plug to cooperate with the processor. The device also includes an active bridge rectifier connected with the audio plug adapted to minimize voltage drop between the pressure transducer, the processor, and the audio plug. The device further includes a linear voltage regulator connected with the active bridge rectifier and adapted to regulate output of the active bridge rectifier. In certain embodiments, the device includes an amplifier that is connected with the pressure transducer and is adapted to amplify the pressure signal from the pressure transducer.

The present disclosure further relates to methods for assessing a patient's oral motor performance. The method includes inserting a pressure transducer in patient's mouth to obtain a pressure signal when the patient applies pressure to the pressure transducer. The pressure transducer is configured to convert the pressure signal into voltage that Is proportional to pressure. The method further includes using a processor to take pressure signal samples from the pressure transducer and to convert the pressure signal to a digital pressure value. The digital pressure value is transmitted to a computer where it is displayed. In certain embodiments, the computer is a smartphone that includes an audio jack. In certain embodiments, the method includes connecting an amplifier to the pressure transducer to amplify the pressure signal. In other embodiments, the method includes connecting an audio plug to the processor and the audio jack of the smartphone and transmitting the digital pressure value through a microphone input of the smartphone. In yet other embodiments, the method includes saving the digital pressure value in a memory device and associating the digital pressure value with a stored record of a patient. The method may further include having the patient apply pressure on the pressure transducer using his tongue for a predetermined amount of time and recording the digital pressure value as an indication of the tongue strength of the patient. In certain embodiments, the method includes setting a target digital pressure value, having the patient apply pressure on the pressure transducer, and providing a display showing the digital pressure value generated by the patient and amount of time the patient has been maintaining the pressure on the pressure transducer that generates the target digital pressure. The method may also include stepping up the voltage of the audio plug, using a rectifier to minimize voltage drop between the pressure transducer, the processor, and the audio plug, and regulating the voltage of the rectifier.

### BRIEF DESCRIPTION OF THE DRAWINGS

FIG. 1 illustrates one embodiment of a system for monitoring and promoting oral motor strength.

FIG. 2 is a schematic diagram of one implementation of the system of FIG. 1.

FIG. 3A is a schematic diagram of another implementation of the system of FIG. 1.

FIGS. 3B1 and 3B2 are schematic diagrams of yet another implementation of the system of FIG. 1.

FIG. 4 is a schematic diagram of another implementation of the device of FIG. 1.

FIG. 5 is a flowchart showing operation of the system or method of assessing a patient's oral motor performance.

FIG. 6 is a screenshot of a loading page of a software program executed by the mobile device.

FIG. 7 is a terms and condition page of the software program.

FIG. 8 is a log-in page of the software program.

FIG. 9 is a main menu screen of the software program operating in clinician mode.

FIG. 10 is a list of patients screen of the software program.

FIG. 11 is an individual patient data screen of the software program.

FIG. 12 is the main menu screen of the software program with the start icon being selected.

FIG. 13 is a mode selection screen of the software program.

FIG. 14 is an exercise selection screen of the software program.

FIG. 15 is pressure meter screen showing the digital pressure value exerted by the patient, a timer, and a start/reset button.

FIG. 16 is the exercise selection screen of FIG. 14 with the tongue endurance exercise being selected.

FIG. 17 is the pressure meter screen before the tongue endurance exercise.

FIG. 18 is the pressure meter screen during the tongue endurance exercise.

FIG. 19 is the mode selection screen with the evaluation/therapy mode selected.

FIG. 20 is the evaluation/therapy screen.

FIG. 21 is the begin therapy screen.

FIG. 22 is the pre-tongue endurance therapy screen showing a target digital pressure value.

FIG. 23 is a list of results screen.

FIG. 24 is an individual result screen.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

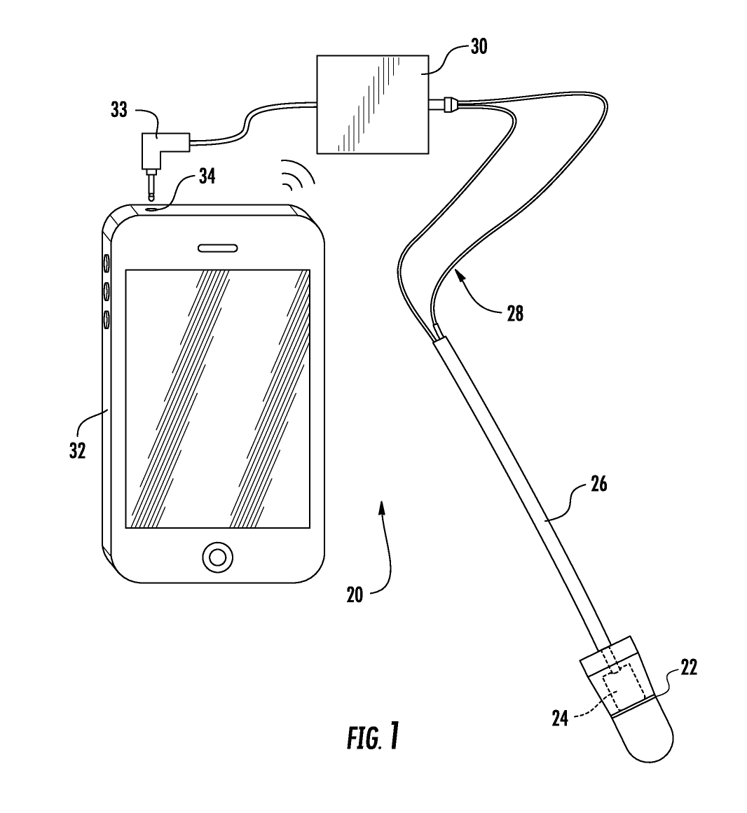

FIG. 1 shows a system 20 for monitoring and promoting the strength and endurance of a patient's oral motor system, including the tongue, lips, jaw, and the mouth. System 20 includes a flexible pressure bulb 22 that houses a pressure transducer 24. The flexible pressure bulb 22 is inserted into the patient's mouth, and the patient applies pressure using either the upper and lower lips to obtain an indication of the lip strength. The patient may also apply pressure using the tongue against the palate to obtain an indication of the user's tongue strength. The pressure transducer 24 may be a piezoelectric pressure sensor, such as a quartz crystal, with a horizontal shaft attached to the quartz crystal and the flexible pressure bulb. Whenever the flexible pressure bulb receives pressure from the lips or the tongue, the horizontal shaft pressurizes the quartz crystal and produces voltage. The pressure transducer 24 may also be a strain gauge. Voltage would be generated proportional to the compression or expansion of the flexible pressure bulb 22. The transducer signal may be represented by a current or voltage, and may be provided as an analog or digital electrical signal. Transducer 24 may include a power supply, such as a battery, and related circuitry to power the transducer and/or provide various signal conditioning or pre-processing functions. Similarly, transducer 24 may have an associated transmitter, receiver, or transceiver to wirelessly transmit and/or receive data and/or control signals.

In one embodiment, system 20 may include a connector tube 26 and wires 28 that transmit the transducer signal to a processor 30. The processor 30 converts the transducer signal for processing and/or wireless transmission. In one embodiment, processor 30 converts an analog electrical signal from transducer 24 to digital data that may be transmitted wirelessly to a mobile device, such as smartphone 32. Wireless transmission may be performed using any of a number of well-known protocols, such as through a Bluetooth connection or Wi-Fi connection over a local or wide area network, such as the internet. Alternatively, a wired connection may be facilitated using an audio connector 33 connected to the audio jack 34 or other digital or analog input/output interface of the smartphone 32. A computer may be used in lieu of a smartphone 32 including a tablet, desktop, or laptop computer.

Referring to FIG. 2, the processor 30 may be integrated within a microcontroller 36 that converts and processes the transducer signal from the pressure sensor 24 to a signal suitable for communication to a user device. The microcontroller 36 may be powered by a battery 38 and connected to a communications module, implemented by Bluetooth module 40 in this example, so it can send the digital signal to the corresponding receiver of the smartphone as represented by smartphone Bluetooth module 42. In certain embodiments, the smartphone 32 can connect to a network 44, such as a cellular or Wi-Fi network that connects to one or more local or wide area networks such as the internet, and access a server having a software program relating to the diagnosis of dysphagia or an oral motor dysfunction. The digital signal and/or associated data relating to the pressure from the user's lip or tongue can be stored and analyzed by the software program to assess oral motor function.

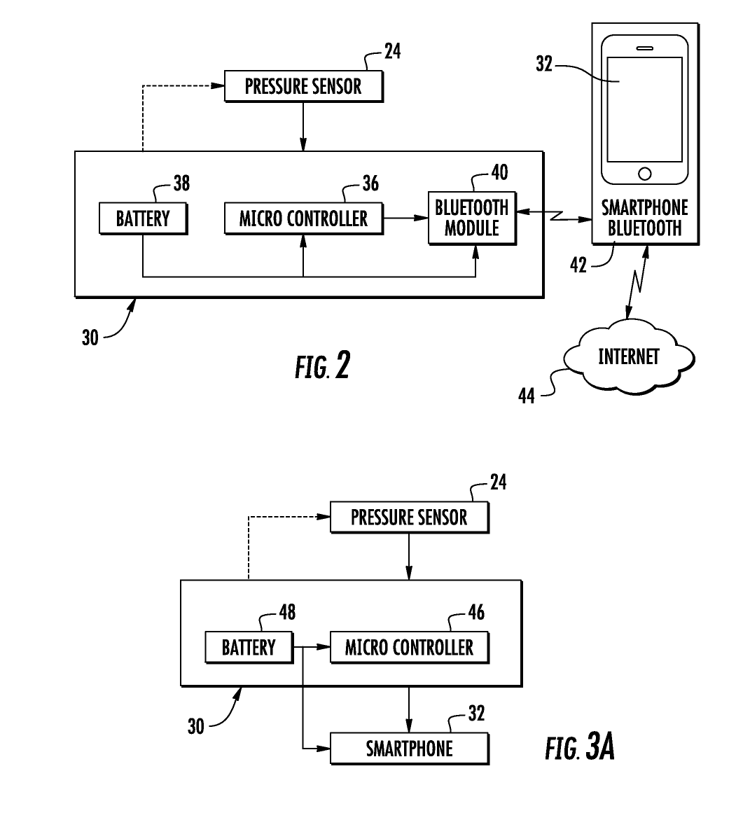

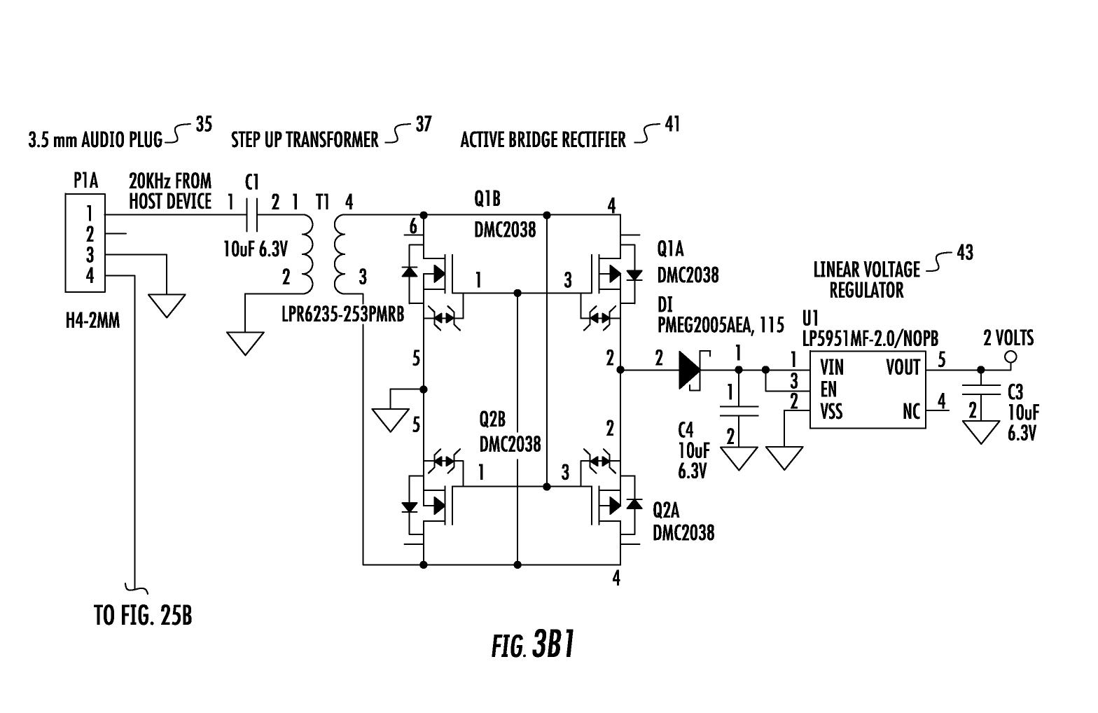

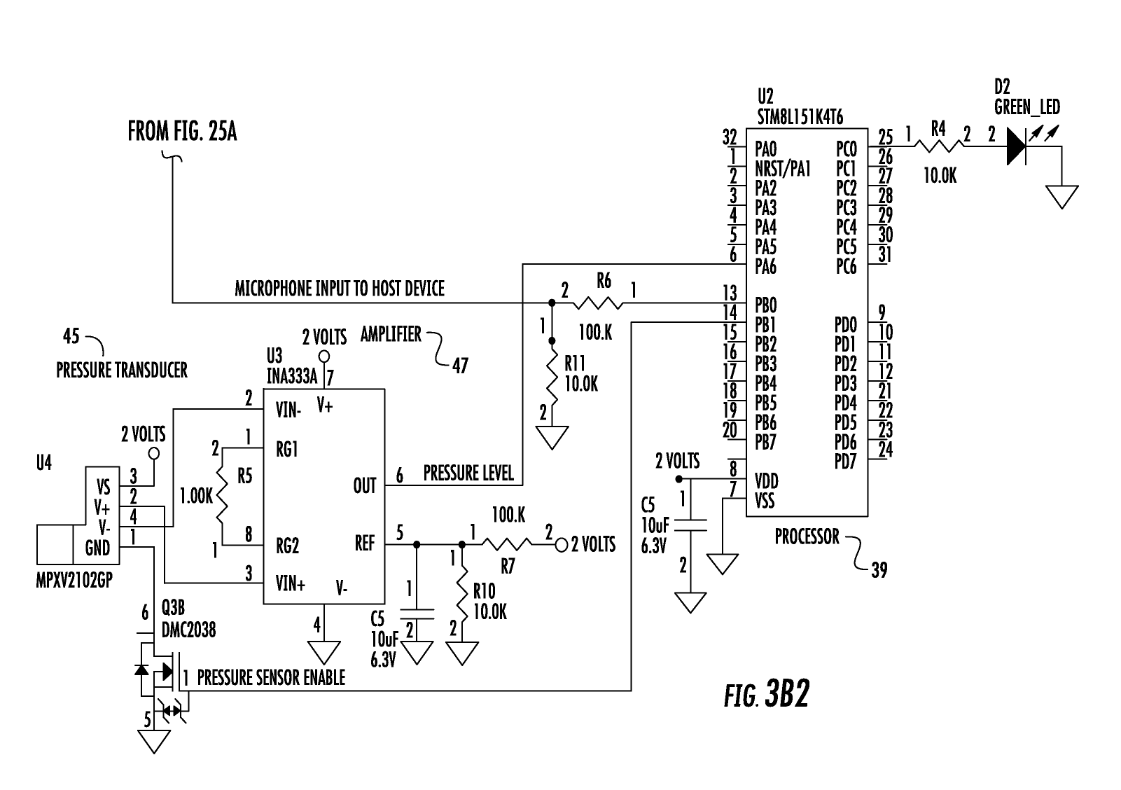

Referring to FIG. 3A, if a wired connection between the processor 30 and the smartphone 32 is preferred over a wireless connection, the processor 30 may be incorporated into a microcontroller 46 that is powered by a battery 48. The microcontroller 46 can use various coding and communication strategies to encode and transmit the transducer signal. For example, microcontroller 46 may use FSK modulation and encode an analog voltage signal to be within similar parameters as an audio signal. The FSK signal is then transmitted through the audio jack 34 of the smartphone 32, and the smartphone can perform FSK demodulation and decode the transducer signal. A mobile application relating to the assessment of oral motor function, monitoring or providing feedback during exercises of patients with oral motor dysfunction is uploaded to the smartphone 32. The mobile application can store the data from the pressure sensor 24, create a patient profile, and store history of pressure measurements of the patient. The mobile application can also provide information used to perform various exercises and assess oral motor function for a patient and provide statistics based on the data from the pressure sensor 24 and show any progress of the patient.

An embodiment using a wired connection is shown in FIG. 3B. A 3.5 mm audio plug 35 is used to connect to a smartphone 32 or a computer. The audio plug 35 has 4 contacts, which are ground, microphone, left audio out, and right audio out. The left and right output lines preferably transmit a full level 20 KHz audio. The audio plug 35 connects to a step up transformer 37, which steps up the voltage from a low level voltage to a higher voltage needed to run the processor 39. An active bridge rectifier 41 is connected to the step up transformer 37 to rectify the 20 KHz signal and avoid or minimize voltage drops. A linear voltage regulator 43 is provided to regulate the output of the bridge rectifier to 2 volts. On the other end, the pressure transducer 45 connects to the pressure sensor 24, which converts the pressure signal into voltage that is proportional to the pressure. The pressure transducer 45 is connected to a processor 39, which can turn on and off the pressure transducer 45 between samples to reduce current requirement. The pressure transducer 45 is also connected to an amplifier 47 to amply the pressure transducer signal to a level that is compatible with the processor's 0 to 2 volt input range. The processor 39 may take pressure signal samples 20 times a second then converts the pressure signal into a frequency that is proportional to the pressure. The converted signal is transmitted to the smartphone 32 or computer through its microphone line. Alternatively, the converted signal can be transmitted through telephone modem signaling, Bluetooth, or through a network connection.

Referring to FIG. 4, the system 20 may include an analog input/output server 50 that receives, converts and processes the analog voltage signal from the pressure transducer 24. A sample analog input/output server is the xSenso from Lantronix, Inc. of Irvine, Calif. The analog input/output server 50 transmits the processed analog voltage signal to the processor 30 via the internet 44 so that a computer 52 or smartphone 32 can receive the signal. The computer 52 or smartphone 32 has a software program relating to the monitoring, analysis, or diagnosis of dysphagia or an oral motor dysfunction. The software program can store the data from the pressure sensor 24, create a patient profile, and history of pressure measurements of the patient. The software program can also provide various resources having information about a treatment plan for a patient and provide statistics based on the data from the pressure sensor 24 and show any progress of the patient.

Referring to FIG. 5, the present disclosure includes a method of obtaining an indication of an oral motor performance as shown in flowchart 60. The method includes obtaining pressure data 62 from a pressure transducer. The pressure data can be obtained by allowing a tongue or a lip to put pressure on a flexible pressure bulb 22 that houses a pressure transducer 24. To measure tongue strength, the patient places the flexible pressure bulb 22 along the hard palate of the mouth just behind the central incisors. The patient then elevates the anterior tongue dorsum to press against the flexible pressure bulb 22 as hard as possible. To measure tongue endurance, the patient places the flexible pressure bulb 22 along the hard palate of the mouth just behind the central incisors. The user then elevates the anterior tongue dorsum to press against the flexible pressure bulb 22 as hard as possible and hold for a predetermined period of time. To measure lip strength, the flexible pressure bulb 22 is positioned inside the patient's cheek just lateral to the corner of the mouth. The patient presses the tongue bulb against his teeth by pursing the lips as hard as he can for about two (2) seconds, then the patient releases his teeth from the tongue bulb.

Next, at step 64, the pressure data is converted or otherwise processed and may be coded for transmission as digital data, such as by the processor 30 or by the analog input/output server 50 described above. At step 68, the transducer digital data is transmitted to a smartphone where a mobile application can be used to store, process, analyze, and use the digital data. The data may be transmitted through the audio jack of the smartphone, or wirelessly using Bluetooth, WiFi or other well-known communication protocols over a local or wide area network such as a cellular network and/or the internet. The digital data may also be transmitted to a server (step 70) that may be accessible via a network. A computer or a smartphone may connect to the network to access the digital data and a software program for storing, processing, analyzing, and using the digital data.

FIGS. 6-8 show several sample screen shots of a mobile application of the present disclosure at a start-up and log-in stage. When the application starts, a loading page 100 with a desired logo 102 is shown (FIG. 6). The next screen 104 has introductory information about the application, legal disclosures, safety, and HIPPAA information (FIG. 7). The user is provided with a choice between a Patient Mode or a Clinician Mode. If the user chooses the Patient Mode, the user is asked to agree that he is using the application and device under the direction of a trained clinician. In the Patient Mode, the user will be allowed to save and access his personal data, goals, and progress. The user will not have access to any other user or patient data. In the Clinician Mode, the user will have to option to save and access the data, goals, and progress of multiple users or patients. After providing the introductory information and desired disclosures, a log-in 106 screen is displayed (FIG. 8).

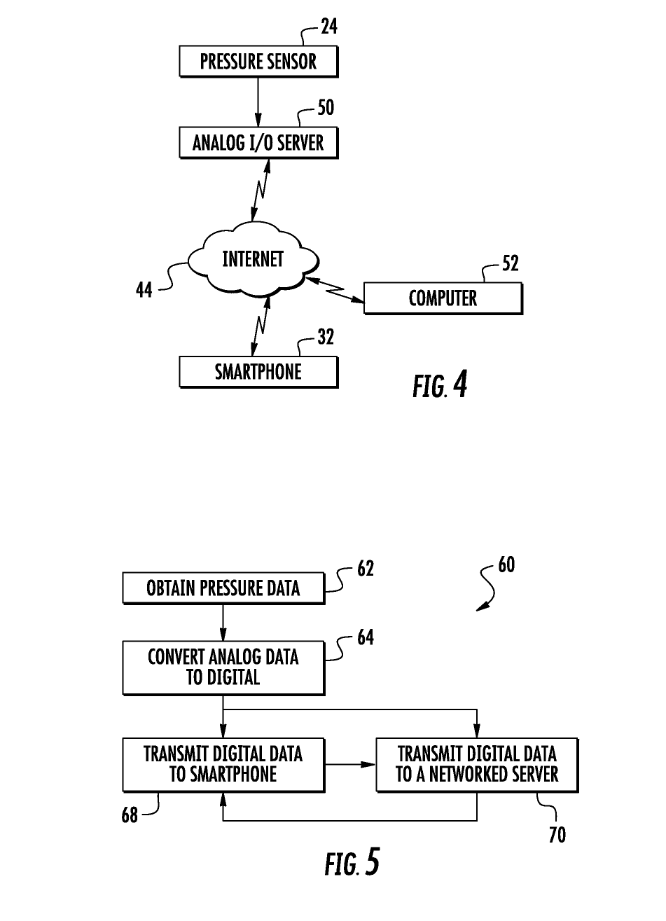

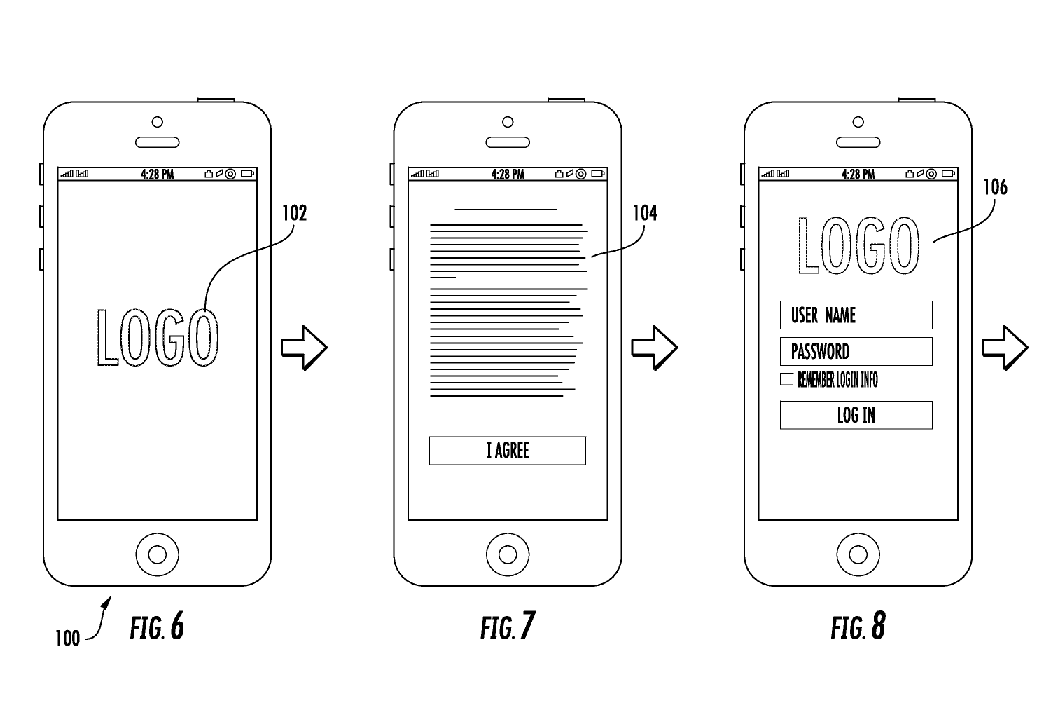

FIG. 9 shows a main menu screen 108 in Clinician Mode. The main menu screen 108 includes a “My Patients” icon 110, which allows the user to see a database of patients and to add, remove, or modify patients' information. The main menu screen 108 also includes a “Demo Video” icon 112, which allows access to an animation of instructions on how to set-up or use the system. A “Start” icon 114 is also provided and allows the user to start a practice mode, an evaluation mode, or a therapy mode. FIG. 10 shows a patient database screen 116, which is shown after the “My Patients” icon 110 is pressed and shows all the saved patients' information. There is an “Add Patient” icon 118 that allows the user to add a new patient's information. FIG. 11 shows a detailed patient information screen 120, which is displayed once a patient is selected from the patient database screen 116. The detailed patient information screen 120 includes a graph of the patients' oral motor performance progress during the course of multiple practice, evaluation, or therapy sessions.

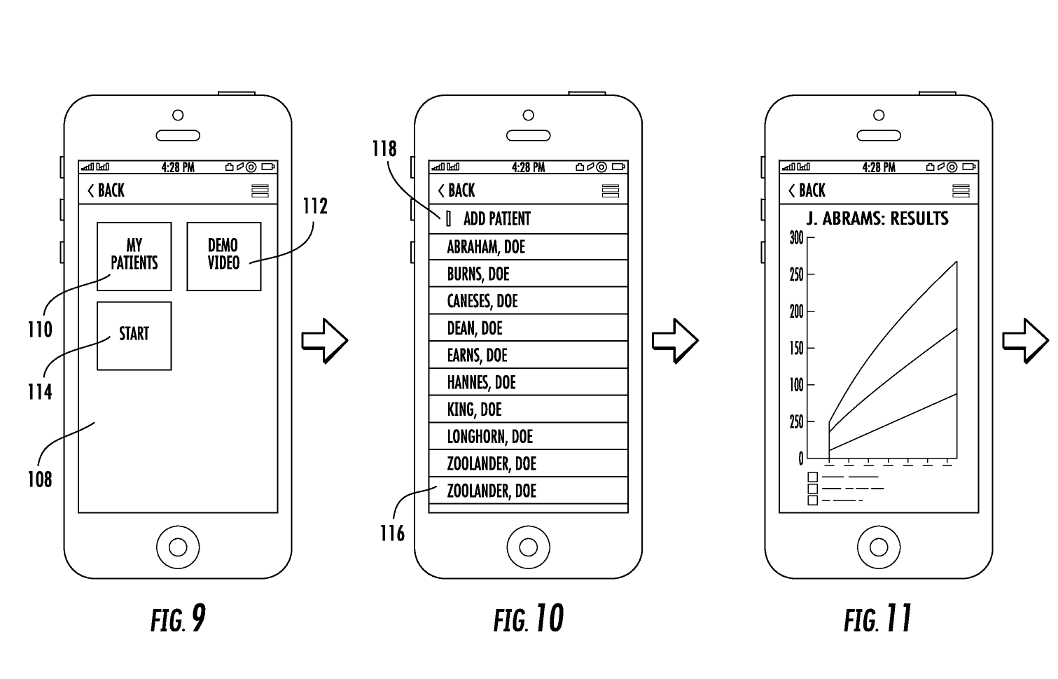

In FIG. 12, the “Start” icon 112 is pressed. A mode selection screen 122 follows, where the user is asked whether to run a practice mode 124 or an evaluation/therapy mode 125 (FIG. 13). An exercise selection screen 128 follows and prompts the user to select whether to measure the tongue strength 130, the tongue endurance 132, or the lip strength 134 (FIG. 14). To measure tongue strength, the user places the flexible pressure bulb 22 along the hard palate of the patient's mouth just behind the central incisors. The patient then elevates the anterior tongue dorsum to press against the flexible pressure bulb 22 as hard as possible. To measure tongue endurance, the user places the flexible pressure bulb 22 along the hard palate of the patient's mouth just behind the central incisors. The patient then elevates the anterior tongue dorsum to press against the flexible pressure bulb 22 as hard as possible and hold for a predetermined period of time. To measure lip strength, the flexible pressure bulb 22 is positioned inside the patient's cheek just lateral to the corner of the mouth. The patient presses the tongue bulb against his teeth by pursing the lips as hard as he can for about two (2) seconds, then the patient releases his teeth from the tongue bulb. The user may choose to go back to the main menu screen 108 and play a demo video for guidance.

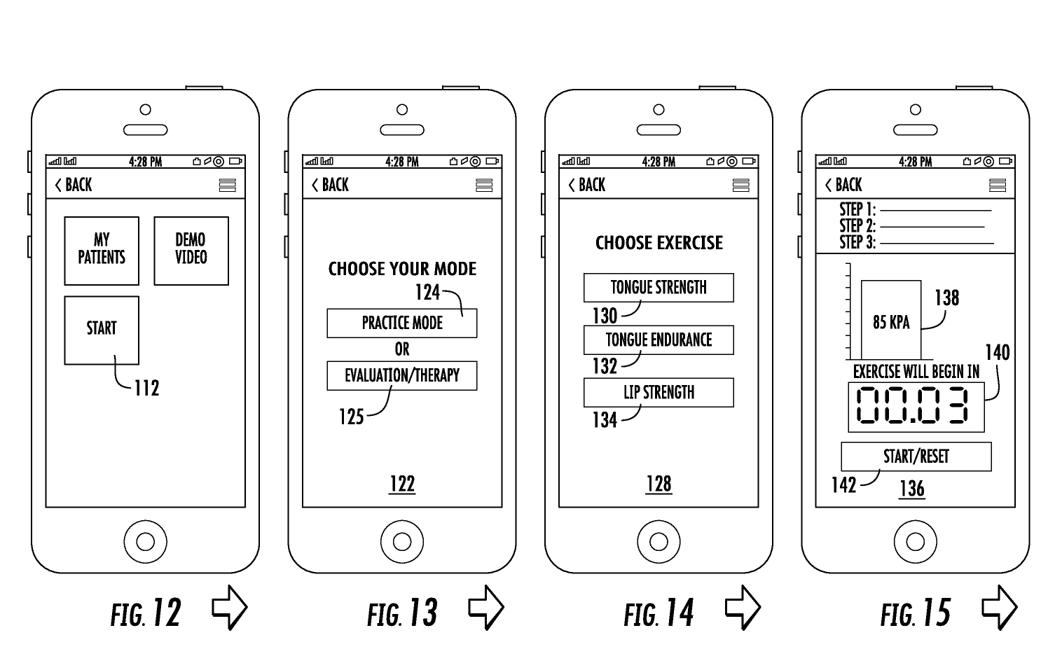

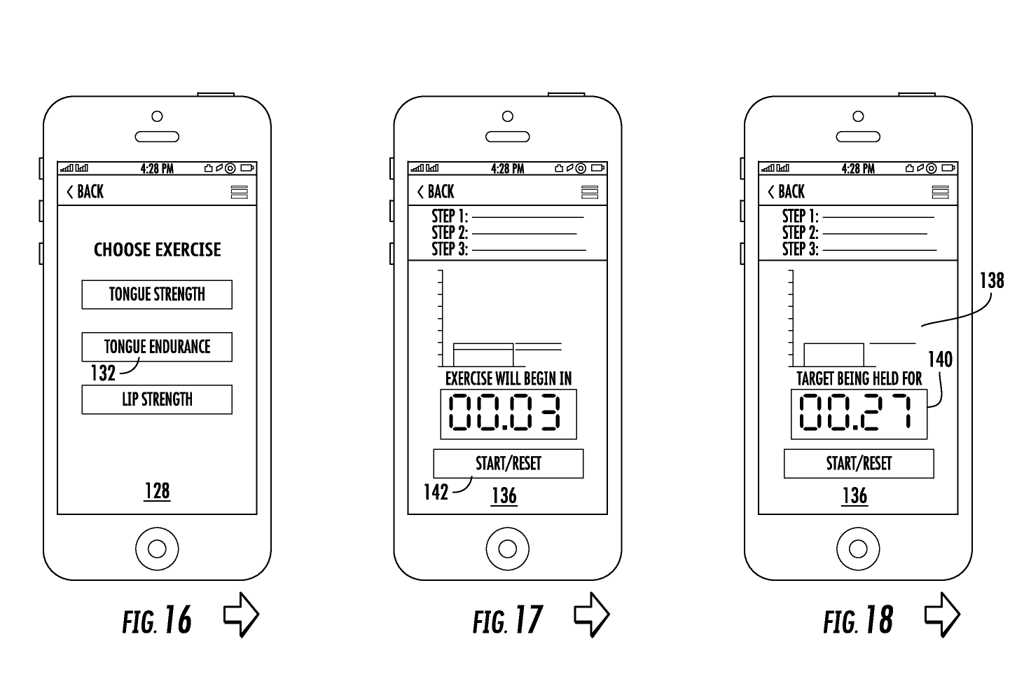

FIG. 15 shows a pressure meter screen 136 where a pressure reading is displayed as a graph 138 with 0-100 kPA on a y-axis, time unit on the x-axis, and a digital readout of the pressure reading in kPA. A timer 140 is also provided and indicates when the next exercise will begin. A start/reset icon 142 is provided to allow the user to indicate when to start saving data or whether to discard the data. FIG. 16 shows the exercise selection screen 128 with the tongue endurance icon 132 being selected. In FIG. 17, the pressure meter screen 136 is shown again. The user can press the start/reset icon 142. Once the start icon is pressed, the timer counts down 3, 2, 1 and the patient presses and holds the pressure bulb as long as the patient can. In FIG. 18, the graph 138 of pressure meter screen 136 can be used as a target indicator for a patient. The timer 140 can also show the patient how long he has maintained applying the target pressure. The timer 140 will display the final time at the end of the exercise and notifies the patient the total amount of time he has maintained applying the target pressure. After the exercise, the user may choose to repeat the current practice, move to the next practice, or to go to Evaluation/Therapy mode.

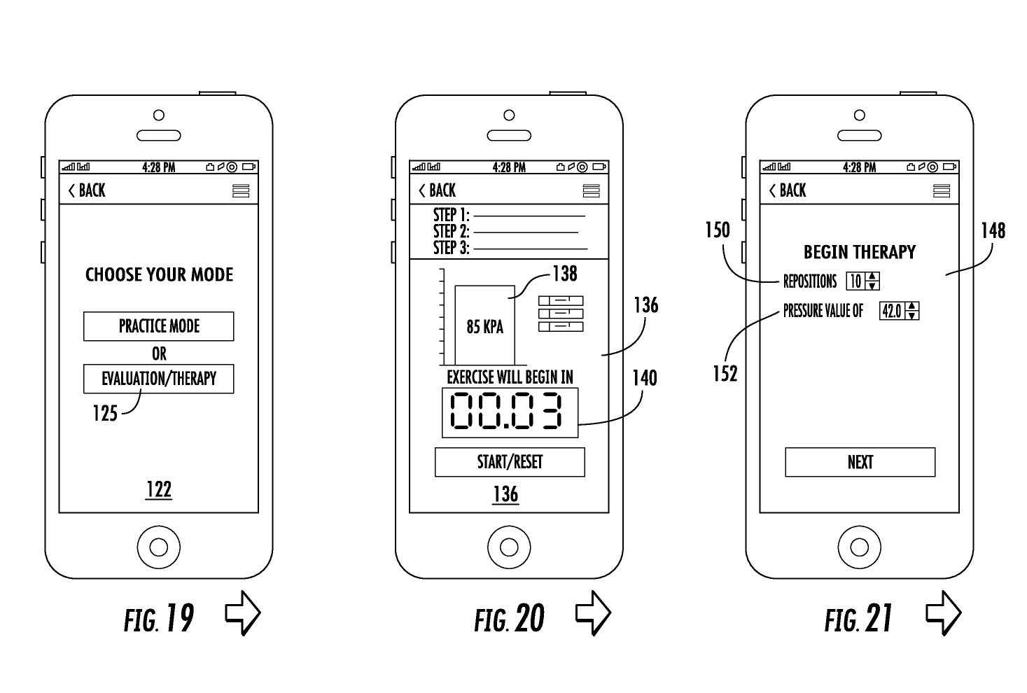

FIG. 19 shows the mode selection screen 122 with the evaluation/therapy mode 125 selected. The application first conducts the evaluation mode then the therapy mode. In the evaluation mode, the patient is evaluated for tongue strength, tongue endurance, then for lip strength. To measure tongue strength, the user places the flexible pressure bulb 22 along the hard palate of the patient's mouth just behind the central incisors. The patient then elevates the anterior tongue dorsum to press against the flexible pressure bulb 22 as hard as possible. In FIG. 20, the pressure meter screen 136 provides a pressure reading through the graph 138. In between pressure readings, a rest period such as 30 seconds can be set. The timer 140 can be used to tell the user and the patient when the next pressure reading will be taken and when the patient will have to press against the flexible pressure bulb 22 again. The tongue strength can be evaluated by taking three pressure readings and taking the highest pressure reading out of the three attempts.

Based on the highest pressure reading from the tongue strength evaluation, tongue endurance can be measured. For instance, if the user wants to evaluate the tongue endurance at 50% of the highest tongue strength reading and the highest pressure reading from the tongue strength is 85 kPA, the amount of time the patient can hold and maintain at least 42.5 kPA would be the tongue endurance. The value of the pressure target can be adjusted to 50-80% of the highest tongue strength reading. The user places the flexible pressure bulb 22 along the hard palate of the patient's mouth just behind the central incisors. The user then elevates the anterior tongue dorsum of the patient to press against the flexible pressure bulb 22 as hard as possible and tells the patient to hold. The user the graph 138 of pressure meter screen 136 can be used as a target indicator for a patient. The timer 140 can also show the patient how long he has maintained applying the target pressure. The timer 140 will display the final time at the end of the evaluation and notifies the patient the total amount of time he has maintained applying the target pressure. The final time and the target pressure will be the indication of tongue endurance.

To measure lip strength, the flexible pressure bulb 22 is positioned inside the patient's cheek just lateral to the corner of the mouth. The patient then will press the tongue bulb against his teeth by pursing the lips as hard as he can for about two (2) seconds, then the patient will release his teeth from the tongue bulb. In FIG. 20, the pressure meter screen 136 provides a pressure reading through the graph 138. In between pressure readings, a rest period such as 30 seconds can be set. The timer 140 can be used to tell the user and the patient when the next pressure reading will be taken and when the patient will have to press against the flexible pressure bulb 22 again. The lip strength can be evaluated by taking three pressure readings and taking the highest pressure reading out of the three attempts.

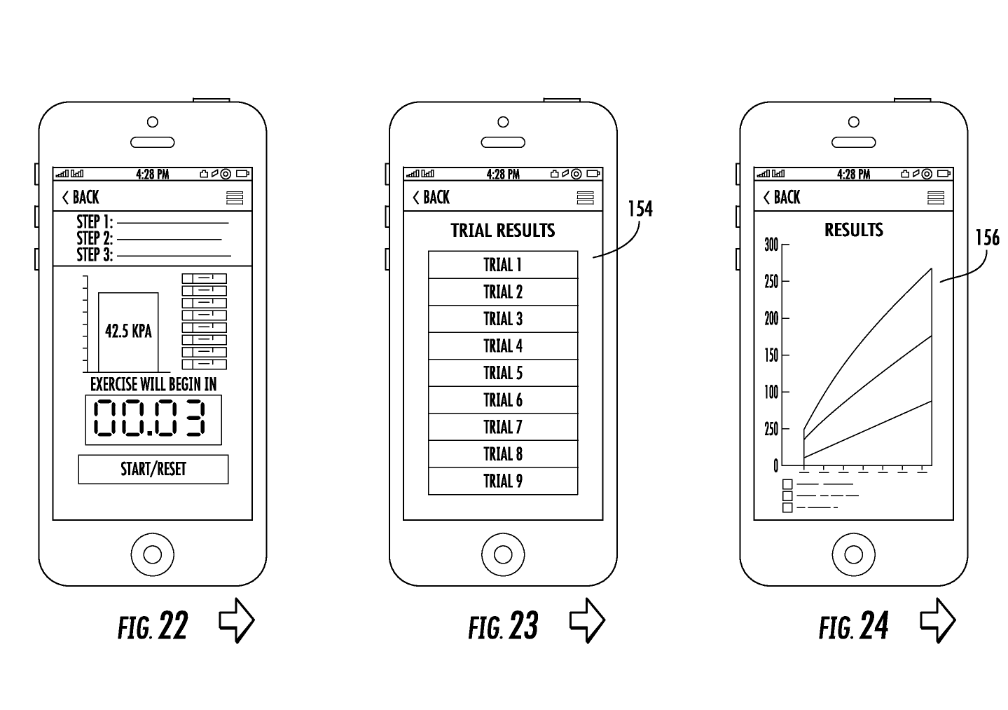

FIG. 21 shows the therapy mode screen 148. The user can set the number of repetitions 150 the user wants to do either the tongue strength, tongue endurance, or lip strength exercises. The user can also set a target pressure value 152 for the exercises. At the end of the therapy session, the user can choose whether to repeat the therapy, change therapy settings, or end the therapy. If end therapy is selected, the user will be prompted to record the data gathered during the therapy. The data recorded will be associated with the patient in the patient database. The user may be given the option to e-mail or share the data. FIGS. 22-24 show additional screen shots when a user goes over a patient's data. FIG. 22 shows a pressure meter screen showing a pressure reading from a prior practice, evaluation or therapy session. FIG. 23 shows a results screen 154 listing each tongue strength, tongue endurance, or lip strength practice, evaluation or therapy reading taken. When a listing is selected, a detailed view can be shown and that includes the pressure reading, the parameters, the date when the reading was taken, and any notes or clinical observations the user may have. FIG. 24 shows a progress screen 156 where the system compiles a predefined set of past data and allows a user can keep track of a patient's oral motor performance progress.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A system for obtaining an indication of an oral motor performance comprising:
a pressure transducer adapted to be positioned within a patient's mouth and adapted to generate a transducer signal in response to pressure applied by the patient;
a processor adapted to receive and process the transducer signal; and
a mobile device in communication with the processor adapted to receive the processed transducer signal and display corresponding pressure unit of the processed transducer signal.

2. The system of claim 1 further comprising a Bluetooth module connected with the processor and adapted to send the processed transducer signal to the mobile device via airwaves.

3. The system of claim 1 wherein the mobile device receives the processed transducer signal through an audio jack.

4. The system of claim 1 further comprising an analog input/output server adapted to receive the transducer signal from the pressure transducer and transmit the transducer signal to a network, the mobile device receiving the transducer signal through the network.

5. The system of claim 1 wherein the processor conducts FSK modulation to encode the transducer signal as audio.

6. The system of claim 5 wherein the mobile device conducts FSK demodulation to decode the transducer signal as audio.

7. An oral motor performance assessment and therapy device comprising:
a pressure transducer adapted to obtain a pressure signal from a patient's mouth;
a processor connected to the pressure transducer and adapted to obtain the pressure signal from the pressure transducer and convert the pressure signal to a digital pressure value;
an audio plug connected to the processor; and
a smartphone connected to the audio plug, the audio plug adapted to transmit the digital pressure value to the smartphone, the smartphone configured to display and store the digital pressure value.

8. The device of claim 7 further comprising an amplifier connected with the pressure transducer and adapted to amplify the pressure signal from the pressure transducer.

9. The device of claim 7 further comprising a step up transformer connected with the audio plug adapted to step up voltage of the audio plug to cooperate with the processor.

10. The device of claim 7 further comprising an active bridge rectifier connected with the audio plug adapted to minimize voltage drop between the pressure transducer, the processor, and the audio plug.

11. The device of claim 10 further comprising a linear voltage regulator connected with the active bridge rectifier and adapted to regulate output of the active bridge rectifier.

12. The device of claim 7 further comprising:
a step up transformer connected with the audio plug adapted to step up voltage of the audio plug to cooperate with the processor;
an active bridge rectifier connected with the audio plug adapted to minimize voltage drop between the pressure transducer, the processor, and the audio plug; and
a linear voltage regulator connected with the active bridge rectifier and adapted to regulate output of the active bridge rectifier.

13. The device of claim 7 wherein the processor is configured to operate with a sampling rate of at least 20 kHz.

14. The device of claim 7 wherein the digital pressure value is transmitted through a microphone input of the smartphone.

15. A method of assessing a patient's oral motor performance comprising:
inserting a pressure transducer in patient's mouth to obtain a pressure signal when the patient applies pressure to the pressure transducer, the pressure transducer configured to convert the pressure signal into voltage that is proportional to pressure;
using a processor to take pressure signal samples from the pressure transducer and convert the pressure signal to a digital pressure value;
transmitting the digital pressure value to a computer; and
displaying the digital pressure value on the computer.

16. The method of claim 15 wherein the computer is a smartphone that includes an audio jack, the method further comprising:
connecting an amplifier to the pressure transducer to amplify the pressure signal;
connecting an audio plug to the processor and the audio jack of the smartphone; and
transmitting the digital pressure value through a microphone input of the smartphone.

17. The method of claim 16, further comprising:
stepping up the voltage of the audio plug;
using a rectifier to minimize voltage drop between the pressure transducer, the processor, and the audio plug; and
regulating the voltage of the rectifier.

18. The method of claim 15 further comprising saving the digital pressure value in a memory device and associating the digital pressure value with a stored record of a patient.

19. The method of claim 15 further comprising having the patient apply pressure on the pressure transducer using his tongue for a predetermined amount of time and recording the digital pressure value as an indication of tongue strength of the patient.

20. The method of claim 15 further comprising setting a target digital pressure value, having the patient apply pressure on the pressure transducer, and providing a display showing the digital pressure value generated by the patient and amount of time the patient has been maintaining the pressure on the pressure transducer that generates the target digital pressure.
